(19) United States
(12) Patent Application Publication
(10) Pub. No.: US 2007/0083248 A1
(43) Pub. Date: Apr. 12, 2007

(54) Healthful belt

(76) Inventor: Yu-Ting Tu, Tainan City (TW)

Correspondence Address: Yu-Ting Tu, P.O. Box 90, Tainan City, 70499, TW

(21) Appl. No.: 11/246,054

(22) Filed: Oct. 11, 2005

(51) Int. Cl. A61F 7/00

(52) U.S. Cl. 607/108; 607/112

(57) ABSTRACT

A healthful belt mainly has plural silicone rubber projections possessing far-infrared energy on the surface of an elastic cloth. When the belt is worn on or wrapped around the sore spot, the silicone rubber projections is to contact with the skin directly, the far-infrared energy sinking into the skin organization and a knee so as to improve blood circulation, metabolism and immunity function, diminishing fatigue and pain and helpful to health of human bodies.

### BACKGROUND OF THE INVENTION

1. Field of the Invention

This invention relates to a healthful belt, particularly to one that is composed of a belt body and plural silicone rubber projections having far-infrared energy set on a surface of the belt body, having a far-infrared energy. When the belt is worn on or wrapped around the sore spot if a human body, the silicone rubber projections are to contact with the skin directly, with the far-infrared energy sinking into the skin organization and a knee so as to improve blood circulation, metabolism and immunity function and diminish fatigue and pain, helpful to a person's health.

2. Description of the Prior Art

Nowadays, people often live and work with nervousness and stresses that, with lack of exercise, cause fatigues physically and psychologically and poor blood circulation, resulting in so-called civil diseases. People often get pains with muscle or bones. To soothe such pains, most people are to attach a sore-soothing patch on a sore spot of a human body. But, before attaching the patch on the sore spot, a protective film must be previously torn down. The adhesive mixed with medicines on the patch may be affected in its effect by the temperature change of the environment. Moreover, if the patch is to be attached on the spot located at an elbow or a knee necessary to bend very often, it can not adhere to the skin tightly, causing a much less cure effect. And, when the patch is removed off the skin, it usually loses its adhesive property with torn soft hairs stuck with it, no more able to be re-used and becoming a waste even if there remain available medicines. Therefore, this invention has been devised to offer a healthful belt.

### SUMMARY OF THE INVENTION

The prime object of this invention is to offer a healthful belt provided with far-infrared energy able to improve blood circulation, metabolism and immunity function and diminish fatigue and pain, helpful to health of human bodies.

The main characteristics of the invention are a belt body set with plural silicone rubber projections having far-infrared energy.

### BRIEF DESCRIPTION OF DRAWINGS

This invention is better understood by referring to the accompanying drawings, wherein:

FIG. 1 is a perspective view of a belt of the first embodiment of a healthful belt in this invention;

FIG. 2 is a magnified cross-sectioned view of a belt of the first embodiment of a healthful belt in this invention;

FIG. 3 is a perspective view of the first embodiment in this invention of a healthful belt in the present invention, showing it in a using situation;

FIG. 4 is a perspective view of a second embodiment of a healthful belt in this invention; and

FIG. 5 is a perspective view of the second embodiment of a healthful belt in this invention, showing it in a using situation.

### DETAILED DESCRIPTION OF THE PREFERRED EMBODIMENT

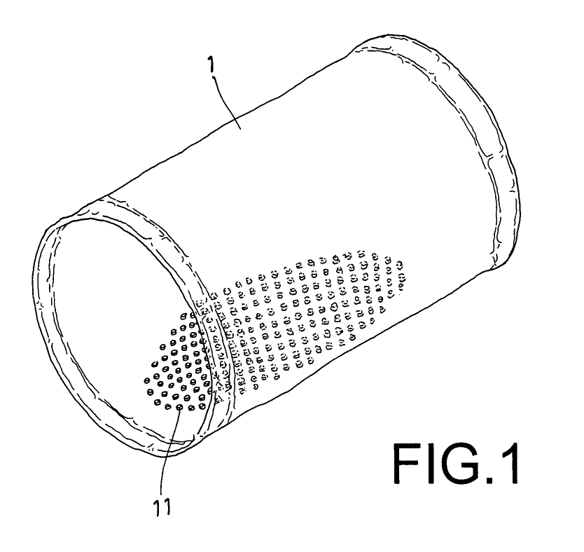

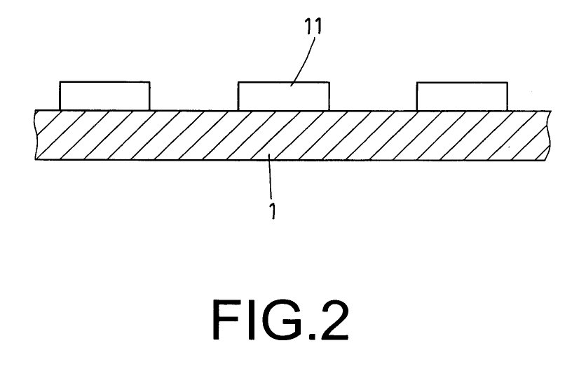

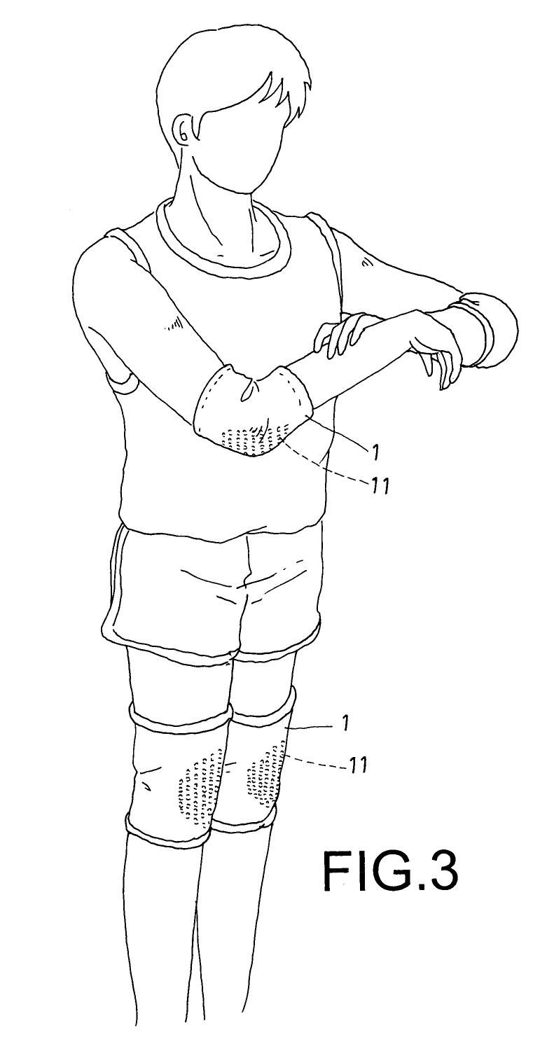

As shown in FIGS. 1 and 2, a preferred embodiment of a healthful belt in the present invention includes a belt body 1 made of an elastic cloth and shaped as a cylindrical protector and plural silicone rubber projections 11 possessing far-infrared energy set on the inner surface of the belt body 1. When the belt body 1 is worn on a sore spot of a human body, such as an elbow or a knee, as shown in FIG. 3, the silicone rubber projections 11 is to contact with the skin directly, with its far-infrared energy sinking into the skin organization or a knee to improve blood circulation, metabolism and immunity function and diminish fatigue and pain, helpful to human health. Moreover, the belt body 1 is very convenient to wear on and take off, usable repeatedly, helping an environmental protection owing to its repeating usability.

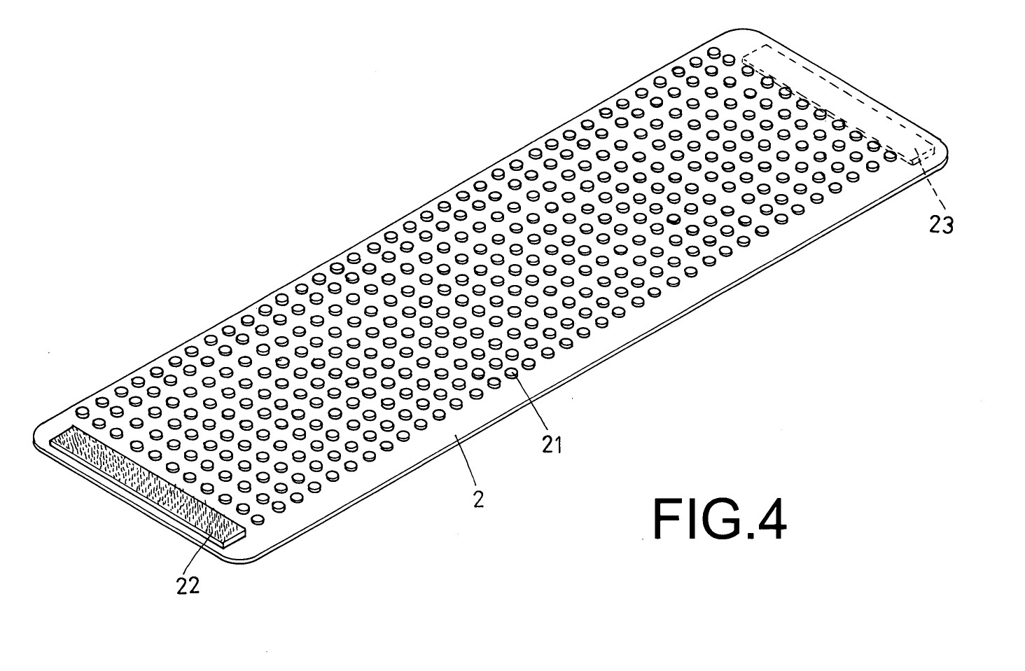

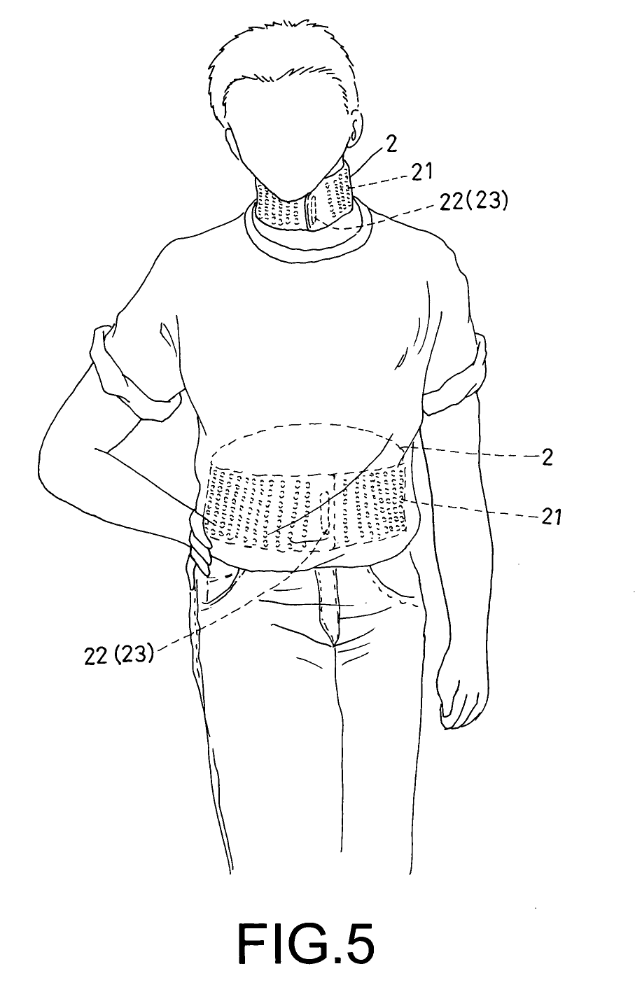

As shown in FIG. 4, a second embodiment of the healthful belt in the present invention includes mainly a belt body 2 shaped rectangular, made of an elastic cloth, and plural silicone rubber projections 21 set at the surface of the belt body 2 and having far-infrared energy, a Velcro band 22 set near to its one end and another Velcro band 23 set at the other surface and near to the other end. When the belt body 2 is wrapped around a sore spot of a human body, such as a waist or a neck, and fixed by attaching mutually the Velcro band 22 and the Velcro band 23, as shown in FIG. 5, the silicone rubber projections 21 is to contact with the skin directly, with its far-infrared energy sinking into the skin organization or a knee to improve blood circulation, metabolism and immunity function and diminish fatigue and pain as the first embodiment does. As the Velcro band 22 and the Velcro band 23 can be pulled to separate from each other, the belt body 2 can be taken off easily for repeating use or washing, helping an environmental protection because of its repeating usability as well.

While the preferred embodiment of the invention has been described above, it will be recognized and understood that various modifications may be made therein and the appended claims are intended to cover all such modifications that may fall within the spirit and scope of the invention.

What is claimed is:

1. A belt comprising:
a belt body formed as a base;
plural silicone rubber projections set on the surface of said belt body and possessing a far-infrared energy.

2. A belt as claimed in claim 1, wherein said belt body is made of an elastic cloth.

3. A belt as claimed in claim 1, wherein said belt body is shaped as a cylindrical protector.

4. A belt as claimed in claim 1, wherein said belt body is shaped rectangular.

5. A belt as claimed in claim 4, wherein said belt body is set with a Velcro band near to its two ends respectively for temporary mutual attachment.
